(19) United States
(12) Patent Application Publication
(10) Pub. No.: US 2008/0051812 A1
(43) Pub. Date: Feb. 28, 2008

(54) Multi-Wire Tissue Cutter

(75) Inventors: Greg Schmitz, Los Gatos, CA (US); Jefferey Bleam, Boulder Creek, CA (US); Jefffrey L. Bleich, Palo Alto, CA (US); Roy Leguidleguid, Union City, CA (US); Vahid Saadat, Saratoga, CA (US)

Correspondence Address: SHAYGLENN LLP, 2755 CAMPUS DRIVE, SUITE 210, SAN MATEO, CA, 94403, US

(73) Assignee: BAXANO, INC., Mountain View, CA (US)

(21) Appl. No.: 11/461,740

(22) Filed: Aug. 1, 2006

(51) Int. Cl. A61B 17/32

(52) U.S. Cl. 606/167

(57) ABSTRACT

A device for cutting tissue in a human body may include an elongate, hollow shaft having a proximal portion and a distal portion, a bundle of flexible wires slidably disposed within at least a portion of the shaft and having a proximal end and a distal end, and an actuator coupled with the proximal portion of the shaft and the proximal end of the bundle of wires. The distal end of the bundle may be configured to facilitate cutting of tissue, and the wires of the bundle may be at least partially free to move, relative to one another, to allow a cross-sectional shape of the bundle to differ along a length from the proximal to the distal end. The actuator may be configured to move the wires back and forth through the hollow shaft to cause the distal ends of the wires to cut tissue.

### BACKGROUND OF THE INVENTION

The present invention relates generally to medical/surgical devices and methods. More specifically, the present invention relates to a multi-wire tissue cutter and methods for making and using same.

A significant number of surgical procedures involve cutting, shaving, abrading or otherwise contouring or modifying tissue in a patient's body. As the demand for less invasive surgical procedures continually increases, performing various tissue modifications such as cutting, contouring and removing tissue often becomes more challenging. Some of the challenges of minimally invasive procedures include working in a smaller operating field, working with smaller devices, and trying to operate with reduced or even no direct visualization of the structure (or structures) being treated. For example, using arthroscopic surgical techniques for repairing joints such as the knee or the shoulder, it may be quite challenging to cut certain tissues to achieve a desired result, due to the required small size of arthroscopic instruments, the confined surgical space of the joint, lack of direct visualization of the surgical space, and the like. It may be particularly challenging in some surgical procedures, for example, to cut or contour bone or ligamentous tissue with currently available minimally invasive tools and techniques. For example, trying to shave a thin slice of bone off a curved bony surface, using a small-diameter tool in a confined space with little or no ability to see the surface being cut, as may be required in some procedures, may be incredibly challenging or even impossible using currently available devices.

Examples of surgical procedures in which bone and other tissues are cut and removed include the various techniques used for treating spinal stenosis. Spinal stenosis occurs when neural tissue and/or neurovascular tissue in the spine become impinged by one or more structures pressing against them, causing one or more symptoms. This impingement of tissue may occur in one or more of several different areas in the spine, such as in the central spinal canal, or more commonly the lateral recesses of the spinal canal and/or one or more intervertebral foramina.

FIGS. 1-3 show various partial view of the lower (lumbar) region of the spine. FIG. 1 shows an approximate top view of a vertebra with the cauda equina (the bundle of nerves that extends from the base of the spinal cord through the central spinal canal) shown in cross section and two nerve roots exiting the central spinal canal and extending through intervertebral foramina on either side of the vertebra. The spinal cord and cauda equina run vertically along the spine through the central spinal canal, while nerve roots branch off of the spinal cord and cauda equina between adjacent vertebrae and extend through the intervertebral foramina. Intervertebral foramina may also be seen in FIGS. 2 and 3, and nerves extending through the foramina may be seen in FIG. 2.

One common cause of spinal stenosis is buckling and thickening of the ligamentum flavum (one of the ligaments attached to and connecting the vertebrae), as shown in FIG. 1. (Normal ligamentum flavum is shown in cross section in FIG. 3) Buckling or thickening of the ligamentum flavum may impinge on one or more neurovascular structures, dorsal root ganglia, nerve roots and/or the spinal cord itself. Another common cause of neural and neurovascular impingement in the spine is hypertrophy of one or more facet joints (or “zygopophaseal joints”), which provide articulation between adjacent vertebrae. (Two vertebral facet superior articular processes are shown in FIG. 1. Each superior articular process articulates with an inferior articular process of an adjacent vertebra to form a zygopophaseal joint. Such a joint is labeled in FIG. 3.) Other causes of spinal stenosis include formation of osteophytes (or “bone spurs”) on vertebrae, spondylolisthesis (sliding of one vertebra relative to an adjacent vertebra), facet joint synovial cysts, and collapse, bulging or herniation of an intervertebral disc into the central spinal canal. Disc, bone, ligament or other tissue may impinge on the spinal cord, the cauda equina, branching spinal nerve roots and/or blood vessels in the spine to cause loss of function, ischemia and even permanent damage of neural or neurovascular tissue. In a patient, this may manifest as pain, impaired sensation and/or loss of strength or mobility.

In the United States, spinal stenosis occurs with an incidence of between 4% and 6% of adults aged 50 and older and is the most frequent reason cited for back surgery in patients aged 60 and older. Conservative approaches to the treatment of symptoms of spinal stensosis include systemic medications and physical therapy. Epidural steroid injections may also be utilized, but they do not provide long lasting benefits. When these approaches are inadequate, current treatment for spinal stenosis is generally limited to invasive surgical procedures to remove ligament, cartilage, bone spurs, synovial cysts, cartilage, and bone to provide increased room for neural and neurovascular tissue. The standard surgical procedure for spinal stenosis treatment includes laminectomy (complete removal of the lamina (see FIGS. 1 and 2) of one or more vertebrae) or laminotomy (partial removal of the lamina), followed by removal (or “resection”) of the ligamentum flavum. In addition, the surgery often includes partial or occasionally complete facetectomy (removal of all or part of one or more facet joints). In cases where a bulging intervertebral disc contributes to neural impingement, disc material may be removed surgically in a discectomy procedure.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. In a spinal fusion procedure, the vertebrae are attached together with some kind of support mechanism to prevent them from moving relative to one another and to allow adjacent vertebral bones to fuse together. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Discectomy procedures require entering through an incision in the patient's abdomen and navigating through the abdominal anatomy to arrive at the spine. Thus, while laminectomy, facetectomy, discectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients.

Therefore, it would be desirable to have less invasive methods and devices for cutting, shaving, contouring or otherwise modifying target tissue in a spine to help ameliorate or treat spinal stenosis, while preventing unwanted effects on adjacent or nearby non-target tissues. Ideally, such techniques and devices would reduce neural and/or neurovascular impingement without removing significant amounts of vertebral bone, joint, or other spinal support structures, thereby avoiding the need for spinal fusion and, ideally, reducing the long-term morbidity levels resulting from currently available surgical treatments. In modifying tissue in various parts of the spine, it may often be the case that visualizing the treatment area is difficult, that small spaces and/or tight corners must be navigated, that different types of tissue (e.g., ligament and bone) would ideally be removed, and/or the like. Thus, it may be advantageous to have tissue cutting or modifying devices adapted for such conditions.

It may also be advantageous to have tissue cutting devices capable of treating target tissues in parts of the body other than the spine, while preventing damage of non-target tissues. It may be desirable, for example, to have such cutting devices adapted for various arthroscopic surgical procedures, bone contouring procedures for facial surgery or the like. At least some of these objectives will be met by the present invention.

### SUMMARY OF THE INVENTION

In various embodiments, the present invention provides tissue cutters including multiple wires used to cut tissue or to drive a cutting blade or other cutting mechanism. The tissue cutters are typically at least partially flexible, and the wires in the cutters may enhance flexibility. Generally, a tissue cutter may be configured such that when cutting wires, a cutting blade or the like is in a position for modifying target tissue, one or more sides, surfaces or portions of the tissue cutter configured to avoid or prevent damage to non-target tissue will face non-target tissue.

In various embodiments, during a tissue modification procedure, tensioning or anchoring forces may be applied at or near either or both of a distal portion and a proximal portion of the tissue cutter device, either inside or outside the patient, to urge the tissue cutting surface or portion of the device against target tissue. When anchoring force is applied to one end of a device, for example, pulling or tensioning force may be applied to the unanchored end of the device. In some embodiments, tensioning force may be applied at or near both ends of a device.

In some embodiments, the described methods, apparatus and systems may be used to modify tissue in a spine, such as for treating neural impingement, neurovascular impingement and/or spinal stenosis. In alternative embodiments, target tissues in other parts of the body may be modified.

In one aspect of the present invention, a device for cutting tissue in a human body may include an elongate, hollow shaft having a proximal portion and a distal portion, and a bundle of flexible wires slidably disposed within at least a portion of the shaft. The bundle of wires may have a proximal end and a distal end, where the distal end of the bundle is configured to facilitate cutting of tissue, and where the wires of the bundle are at least partially free to move, relative to one another, to allow a cross-sectional shape of the bundle to differ along a length from the proximal to the distal end. The device may further include an actuator coupled with the proximal portion of the shaft and the proximal end of the bundle of wires, wherein the actuator is configured to move the wires back and forth through the hollow shaft to cause the distal ends of the wires to cut tissue.

In various embodiments, the shaft may have any of a number of different lengths, diameters, configurations and cross-sectional shapes. In some embodiments, the shaft may have one cross-sectional shape along its entire length, while in other embodiments the cross-sectional shape of the shaft may change along its length. Examples of cross-sectional shapes a shaft may have include, but are not limited to, round, square, triangular, oval, elliptical, flat, rectangular, asymmetrical, triangular, v-shaped and w-shaped. In some embodiments, the proximal portion of the shaft has a first cross-sectional shape, and the distal portion of the shaft has a second cross-sectional shape, and the bundle of wires assumes approximately the first cross-sectional shape in the proximal portion and approximately the second cross-sectional shape in the distal portion.

The shaft of the device may have a number of additional characteristics or features in various embodiments. For example, in one embodiments, the shaft proximal portion may be rigid and the shaft distal portion may be at least partially flexible. Optionally, in some embodiments, a flexible distal portion of the shaft may be steerable, and the device may further include at least one shaft steering actuator. In some embodiments, the shaft may include at least one window through which tissue may protrude such that the wires may cut the protruding tissue. Optionally, the shaft may include at least one hollow tissue collection chamber beyond the window. The window may include a blade edge, and the wire bundle may be configured to push tissue against the blade edge. One embodiment may further include a slidable ramp member disposed within the shaft for sliding into contact with the wire bundle to urge at least some of the wires out the window to cut tissue and control a depth of the cut.

In an alternative embodiment, the distal portion of the shaft includes a distal opening, and the wire bundle extends out of the distal opening to cut tissue. Such an embodiment may optionally further include a flexible platform extending beyond the distal opening in the shaft, where the platform extends under the wires to protect non-target tissue.

The wires of the wire bundle may comprise any suitable material, in various embodiments, such as but not limited to nitinol, spring stainless steel or other metallic spring materials. In some embodiments, the wires may be coupled together along at least a portion of their lengths, while in alternative embodiments, the wires may be uncoupled to one another. In one embodiments, the proximal end of each wire includes a coupling member or shape to attach to the actuator, and each wire is individually attached to the actuator. In an alternative embodiment, the bundle of wires may be coupled to the actuator as a unit. In some embodiments, the distal end of the wire bundle itself cuts tissue. In alternative embodiments, the distal end of the wire bundle may be coupled with a blade to cut the tissue. In one embodiment, such a blade may be coupled with the distal end of individual wires in the bundle of wires via individual separate hinges, at separate locations on the blade, such that the blade may move from a first configuration substantially parallel to the path of the wires to a second configuration at an angle to the path of the wires, by separately moving one or more wires coupled with the blade. Optionally, a window on the shaft may include a blade edge, and the blade coupled with the bundle of wires may move toward the blade edge on the window to cut tissue.

In various embodiments of the device, any of a number of suitable actuators may be used. In some embodiments, the actuator may include or consist primarily of a handle. Examples of suitable actuators for use with various embodiments include, but are not limited to, various types of squeezable handles, various types of handles with triggers, ultrasound transducers, and rotary driven reciprocating devices. In one embodiment, the actuator may be capable of pulling, pushing and/or twisting at least one individual wire of the wire bundle, and the wires may be at least partially coupled together, such that the actuator can steer the bundle by manipulating the individual wire(s). Optionally, the wire bundle may further include one or more elongate, flexible members configured to perform a specific task during a tissue cutting procedure. Examples of such elongate, flexible members include, but are not limited to, an optical fiber, a flexible irrigation/suction tube, a flexible high pressure tubing, a flexible insulated tubing for carrying high temperature liquids, a flexible insulated tubing for carrying low temperature liquids, a flexible element for transmission of thermal energy, a flexible insulated wire for the transmission of electrical signals from a sensor, a flexible insulated wire for the transmission of electrical signals towards the distal end of the wires, and an energy transmission wire.

In another aspect of the present invention, a method for cutting tissue in a human body may involve advancing an elongate, hollow shaft of a tissue cutting device at least partway into the body such that a tissue cutting portion of the device faces target tissue and a non-cutting portion of the device faces non-target tissue, and advancing a bundle of flexible, elongate wires longitudinally through the hollow shaft to cut at least a portion of the target tissue using distal ends of the wires.

In some embodiments, advancing the shaft may involve pulling the shaft into place between target and non-target tissue by pulling a guidewire coupled with a distal end of the shaft. In alternative embodiments, advancing the shaft may involve advancing over a guidewire. In some embodiments, advancing the shaft includes positioning a window of the shaft against the target tissue. Optionally, advancing the shaft may further include steering at least a distal, flexible portion of the shaft.

The wires may be advanced through the shaft to cut tissue in a number of different ways, according to various embodiments. In one embodiment, for example, advancing the wires may involve pulling a squeeze handle of a proximal actuator coupled with proximal ends of the wires. In another embodiment, advancing the wires may involve activating an ultrasound transducer coupled with proximal ends of the wires. In yet another embodiment, advancing the wires may involve activating a rotary reciprocating actuator coupled with proximal ends of the wires. Optionally, advancing the wires through the shaft may cause the bundle to change its cross-sectional shape as it passes through differently shaped portions of the shaft.

In some embodiments, advancing the wires may cause at least some of the wires to pass by a window on the shaft to cut tissue protruding through the window. Optionally, advancing the wires may cause some of the wires to extend out of the window. Also optionally, advancing the wires may urge tissue against a sharpened edge of the window to cut tissue. In an alternative embodiment, advancing the wires may cause distal ends of the wires to extend out of a distal opening of the shaft. In some embodiments, advancing the wires may cause the wires to separate at their distal ends. In some embodiments, the distal ends of the wires may be coupled with a blade, and advancing the wires may cause the blade to cut tissue. Alternatively, the distal ends of the wires themselves may cut tissue, without being attached to a blade. In a number of embodiments, the wires may automatically retract after being advanced. Some embodiments of the method include reciprocating the wires back and forth multiple times. Also in some embodiments, advancing the wires may cause at least some cut tissue to pack into a hollow chamber of the shaft.

In addition to cutting tissue by moving back and forth, the bundle of wires may cut tissue in other ways and/or may be used to perform other functions in addition to cutting tissue, according to various embodiments. For example, in one embodiment the method may further include visualizing target tissue with an optical fiber disposed in the bundle of wires. In this or another embodiment, the method may further include introducing and/or suctioning fluid using a flexible tube disposed in the bundle of wires. Some embodiments may involve delivering energy at the distal end of the bundle of wires, using a flexible energy delivery device disposed in the bundle. Some embodiments may involve delivering fluid under high pressure at the distal end of the bundle of wires, using a fluid delivery tube disposed in the bundle. In yet another embodiment, the method may include transmitting electrical signals from a sensor in the distal end of the bundle of wires, using a flexible insulated wire disposed in the bundle.

In another aspect of the present invention, a system for cutting tissue in a human body may include a tissue cutting device and a power source for powering the device. The tissue cutting device may include: an elongate, hollow shaft having a proximal portion with a first cross-sectional shape and a distal portion with a second cross-sectional shape; a bundle of flexible wires slidably disposed within at least a portion of the shaft, each of the wires comprising a proximal end and a distal end, the distal end configured to facilitate cutting of tissue, wherein the wires are sufficiently free to move, relative to one another, to allow a cross-sectional shape of the bundle of wires to change from the first cross-sectional shape of the shaft proximal portion to the second cross-sectional shape of the shaft distal portion; and an actuator coupled with the shaft and the bundle of wires at or near their proximal ends, wherein the actuator is configured to move the wires back and forth through the hollow shaft to cause the distal ends of the wires to cut tissue. The power source may be removably coupled with the actuator to provide power to move the wires back and forth.

In various embodiments, any of a number of suitable actuators and power sources may be used. For example, in one embodiment, the actuator may comprise an ultrasound transducer, and the power source may comprise an ultrasound generator. In an alternative embodiment, the actuator may comprise a rotary driven reciprocating device, and the power source may comprise an electrical power source. In some embodiments, the actuator may include a handle. Optionally, in such embodiments, the power source may be removably coupled with the handle.

These and other aspects and embodiments are described more fully below in the Detailed Description, with reference to the attached Drawings.

### BRIEF DESCRIPTION OF THE DRAWINGS

FIG. 1 is cross-sectional view of a spine, showing a top view of a lumbar vertebra, a cross-sectional view of the cauda equina, and two exiting nerve roots;

FIG. 2 is a left lateral view of the lumbar portion of a spine with sacrum and coccyx;

FIG. 3 is a left lateral view of a portion of the lumbar spine, showing only bone and ligament tissue and partially in cross section;

FIG. 4 is a cross-sectional view of a patient's back and spine with a tissue cutter device in place for performing a tissue removal procedure, according to one embodiment of the present invention;

FIG. 5A is side view of a tissue cutter device, showing blades of the device in an open position, according to one embodiment of the present invention;

FIG. 5B is a side view of the tissue cutter of FIG. 5A, showing the blades in a closed position;

FIG. 5C is a top view of a distal portion of the tissue cutter of FIGS. 5A and 5B, showing the blades in the open position;

FIG. 5D is a top view of the distal portion of FIG. 5C, with the blades in the closed position;

FIG. 5E is a side, cross-sectional view of a portion of the tissue cutter of FIGS. 5A-5D;

FIG. 6 is a perspective view of a portion of a tissue cutter device, according to one embodiment of the present invention;

FIG. 7 is a perspective view of a window portion of a tissue cutter device, according to one embodiment of the present invention;

FIG. 8 is a perspective view of a window portion of a tissue cutter device, according to an alternative embodiment of the present invention;

FIGS. 9A-9F are side views of distal tips of various wires, according to various embodiments of the present invention;

FIGS. 10A-10G are end-on, cross-sectional views of various shafts and wire bundles of various tissue cutter devices, according to various embodiments of the present invention;

FIGS. 11A and 11B are side views of a distal portion of a tissue cutter device including a blade (FIG. 11A) and a bundle of wires (FIG. 11B), according to one embodiment of the present invention;

FIGS. 12A and 12B are side, cross-sectional views of a portion of a tissue cutter device including a ramping mechanism to urge one or more wires out of a window, according to one embodiment of the present invention;

FIG. 13 is a top view of a portion of a tissue cutter device including multiple wires and a radiofrequency wire cutter, according to one embodiment of the present invention;

FIG. 14 is a perspective view of a tissue cutter device including a squeeze handle and rigid and flexible shaft portions, according to one embodiment of the present invention;

FIG. 15 is a perspective view of a tissue cutter device including a rotary drive mechanism, according to one embodiment of the present invention; and

FIG. 16 is a perspective view of a tissue cutter device including an ultrasound drive mechanism, according to one embodiment of the present invention.

### DETAILED DESCRIPTION OF THE INVENTION

Various embodiments of a multiple-wire tissue cutter for modifying tissue in a patient are provided. Although the following description and accompanying drawing figures generally focus on cutting tissue in a spine, in various embodiments, any of a number of tissues in other anatomical locations in a patient may be modified.

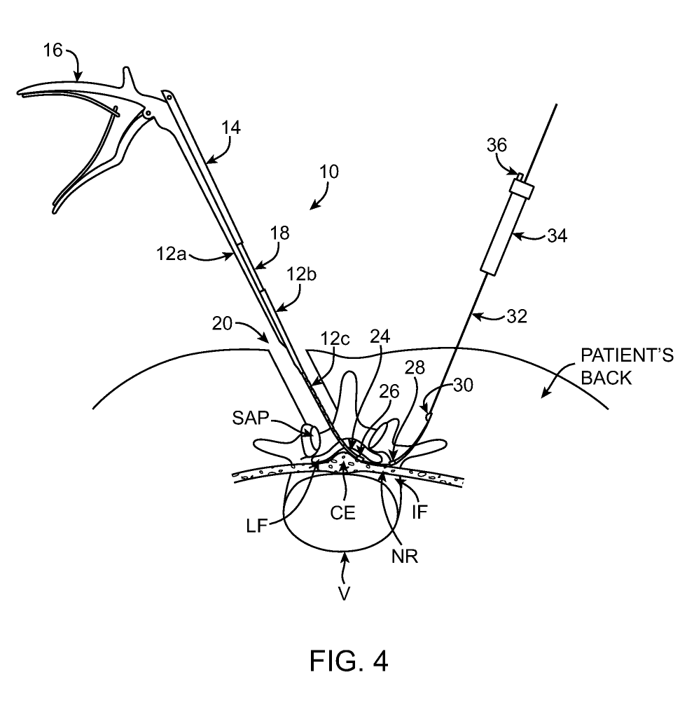

Referring to FIG. 4, one embodiment of a multi-wire tissue cutter device 10 may include a stationary shaft 12 having a proximal rigid portion 12a extending from a proximal handle 16, a distal rigid portion 12b, and a flexible portion 12c. Proximal rigid portion 12a may be coupled with a movable shaft portion 14, and a moveable wire bundle tube 18 may be slidably disposed within distal rigid portion 12b. Distal rigid portion 12b may extend to flatter flexible portion 12c, through which a wire bundle 24 may slidably extend to a proximal blade 26. A platform (or “surface,” “substrate,” or “extension”—not labeled but described in further detail below) may extend from shaft flexible portion 12c and may be coupled with a distal blade 28 and a guidewire connector 30. A tissue cutting system may further include a guidewire 32 and a distal handle 34.

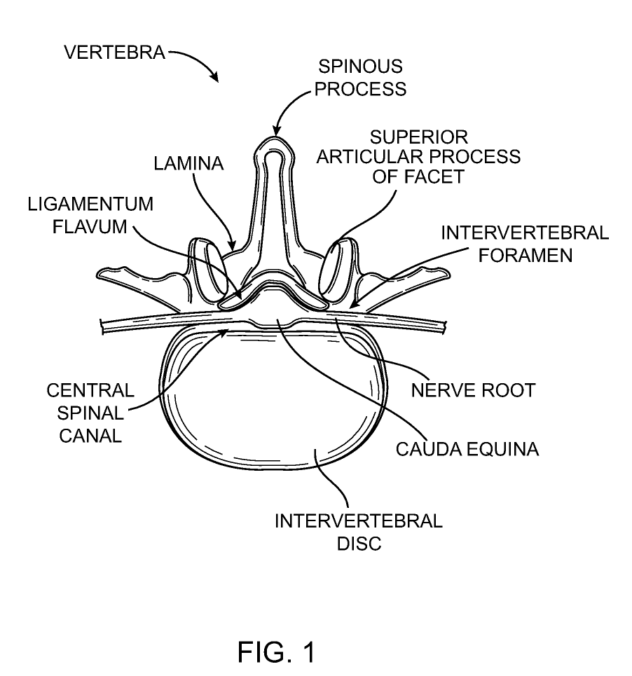

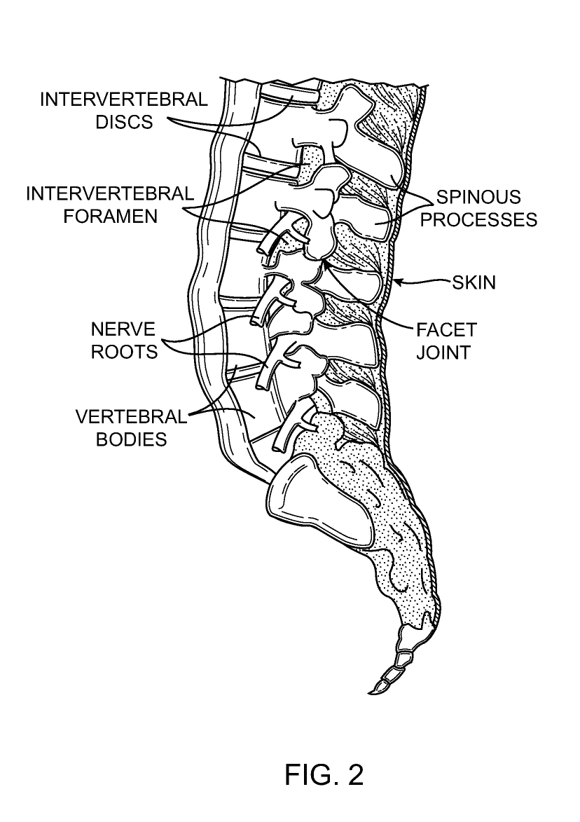

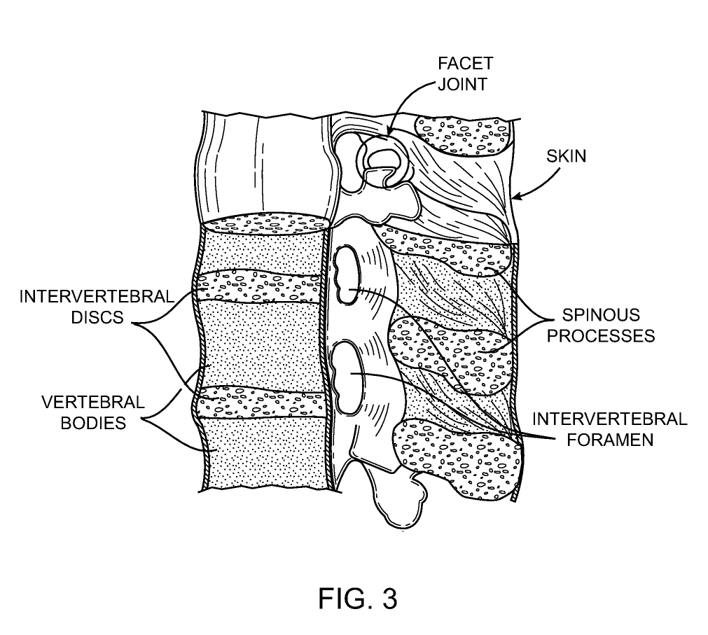

In some embodiments, device 10 may be advanced into a patient's back through an incision 20, which is shown in FIG. 4 as an open incision but which may be a minimally invasive or less invasive incision in alternative embodiments. In some embodiments, device 10 may be advanced by coupling guidewire connector 30 with guidewire 32 that has been advanced between target and non-target tissues, and then pulling guidewire 32 to pull device 10 between the tissues. In alternative embodiments, device 10 may be advanced over guidewire 32, such as via a guidewire lumen or track. The flexibility of flexible portion 12c and the distal extension/platform may facilitate passage of device 10 between tissues in hard-to-reach or tortuous areas of the body, such as between a nerve root (NR) and facet joint and through an intervertebral foramen (IF). Generally, device 10 may be advanced to a position such that blades 26, 28 face tissue to be cut in a tissue removal procedure (“target tissue”) and a non-cutting surface (or surfaces) of device 10 face non-target tissue, such as nerve and/or neurovascular tissue. In the embodiment shown in FIG. 1, blades 26, 28 are positioned to cut ligamentum flavum (LF) and may also cut hypertrophied bone of the facet joint, such as the superior articular process (SAP). (Other anatomical structures depicted in FIG. 1 include the vertebra (V) and cauda equina (CE)).

Before or after blades 26, 28 are located in a desired position, guidewire 32 may be removably coupled with distal handle 34, such as by passing guidewire 32 through a central bore in handle 34 and tightening handle 34 around guidewire 32 via a tightening lever 36. Proximal handle 16 and distal handle 34 may then be used to apply tensioning force to device 10, to urge the cutting portion of device 10 against ligamentum flavum (LF), superior articular process (SAP), or other tissue to be cut. Proximal handle 16 may then be actuated, such as by squeezing in the embodiment shown, which advances moveable shaft 14, thus advancing wire bundle tube 18, wire bundle 24 and proximal blade 26, to cut tissue between proximal blade 26 and distal blade 28. Proximal handle 16 may be released and squeezed as many times as desired to remove a desired amount of tissue. When a desired amount of tissue has been cut, guidewire 32 may be released from distal handle 34, and cutter device 10 and guidewire 32 may be removed from the patient's back.

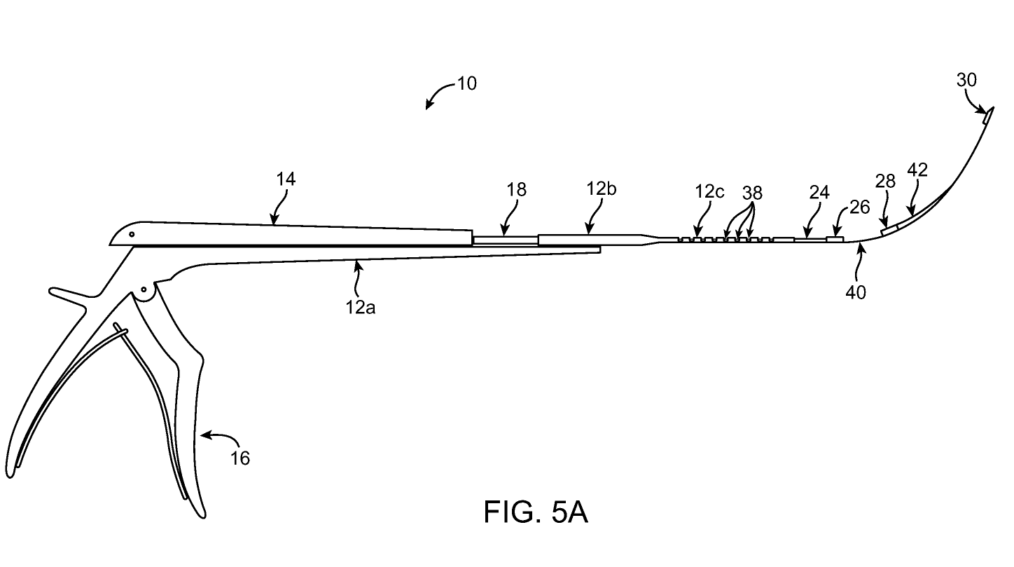

Referring now to FIGS. 5A-5E, tissue cutter device 10 of FIG. 4 is shown in greater detail. In FIG. 5A, a side view of cutter device 10 shows the device structure in greater detail. It can be seen, for example, that distal rigid shaft portion 12b tapers to form flexible shaft portion 12c, which includes multiple slits 38 for enhancing flexibility. Generally, shaft 12 may be formed of any suitable material, such as but not limited to stainless steel. Wire bundle 24 extends through at least part of wire tube 18, through distal rigid portion 12b and flexible portion 12c, and is coupled with proximal blade 26. Wire tube 18 acts to secure the proximal end of wire bundle 24, such as by crimping, welding or the like. In alternative embodiments, wire tube 18 may be excluded, and the proximal end of wire bundle 24 may be otherwise coupled with device. For example, in various embodiments, wire bundle 24 may be coupled with moveable shaft portion 14, may be movably coupled with proximal handle 16, or the like. Extending distally from flexible shaft portion 12c is a platform 40 (or “substrate,” “surface” or “extension”), on which are mounted distal blade 28, a tissue collection chamber 42 and guidewire connector 30. (For the purposes of this application, in various embodiments, the various parts of shaft 12, 14 and platform 40 may be referred to together as the “body” of device 10 or a “device body.”) Collection chamber 42 may be a hollow chamber continuous with distal blade 28, configured such that cut tissue may pass under blade 28, into chamber 42. In this side view, wire bundle 24 appears as a single wire, in this embodiment due to the fact that flattened flexible portion 12c flattens wire bundle 24 to a one-wire-thick cross section. In FIG. 5A, blades 26, 28 are shown in the open position.

In various embodiments, stationary shaft 12 and moveable shaft 14 portions may have any suitable shapes and dimensions and may be made of any suitable materials. For example, in various embodiments, shaft 12, 14 may be made from any of a number of metals, polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. Portions of shaft 12, 14 through which wire bundle 24 travels will generally be predominantly hollow, while other portions may be either hollow or solid. Although one particular embodiment of a shaft mechanism for moving wire bundle 24 is shown, various embodiment may employ any of a number of alternative mechanisms. For example, one embodiment may include a largely or completely flexible shaft, such as an elongate catheter shaft, which extends directly from proximal handle 16. In such an embodiment, wire bundle 24 may couple directly with a drive mechanism of handle 16, so that handle 16 reciprocates wire bundle 24 without employing a rigid shaft structure. In another embodiment, moveable shaft portion 14 may be at least partially hollow, and wire bundle 24 may extend into moveable portion 14 and be attached therein. Therefore, the embodiment of device 10 in FIGS. 4 and 5A-5E is but one example of a multi-wire tissue cutter device. In various alternative embodiments, any of a number of changes made be made to the structure of the device.

As mentioned above, the various components of shaft 12, 14 may have any of a number of shapes. For example, the hollow portions of shaft 12b and 12c, through which wire bundle 24 passes, may have any of a number of cross-sectional shapes in various embodiments. As shown in FIGS. 5A-5E, for example, distal rigid portion 12b may have a round cross-sectional shape, and flexible portion 12c may have a flat shape. In other embodiments, hollow portions 12b, 12c may have one or more other cross-sectional shapes, such as but not limited to round, ovoid, ellipsoid, flat, cambered flat, rectangular, square, triangular, symmetric or asymmetric cross-sectional shapes. In another alternative embodiment, a hollow portion of a shaft may have a continuous cross-sectional shape along its entire length. In some embodiments, at least a distal portion of shaft 12, 14 may have a small profile, to facilitate passage of that portion into a patient, through an introducer device, between target and non-target tissues, through one or more small anatomical channels and/or around an anatomical curve with a small radius of curvature. In some embodiments, for example, shaft 12, 14 may have a height of not more than about 10 mm at any point along its length and a width of not more than about 20 mm at any point along its length, or more preferably a height not more than about 5 mm at any point along its length and a width of not more than about 10 mm at any point along its length, or even more preferably a height not more than about 2 mm at any point along its length and a width of not more than about 4 mm at any point along its length. Shaft flexible portion 12c generally has a configuration and thickness to provide some amount of flexibility, and its flexibility may be further enhanced by one or more slits 38 in the shaft material. Any number and width of slits 38 may be used, in various embodiments, to confer a desired amount of flexibility.

In various embodiments, platform 40 may comprise an extension of a surface of shaft flexible portion 12c. Alternatively, platform 40 may comprise one or more separate pieces of material coupled with shaft flexible portion 12c, such as by welding or attaching with adhesive. Platform 40 may comprise the same or different material(s) as shaft 12, according to various embodiments, and may have any of a number of configurations. For example, platform 40 may comprise a flat, thin, flexible strip of material (such as stainless steel), as shown in FIG. 5A. In an alternative embodiment, platform 40 may have edges that are rounded up to form a track through which proximal blade 26 may travel. Platform 40 will typically be flexible, allowing it to bend, as shown in FIG. 5A. In some embodiments, platform 40 may be made of a shape memory material and given a curved shape, while in other embodiments, platform 40 may be rigid and curved or rigid and straight. Differently shaped platforms 40 and/or platforms 40 having different amounts of flexibility may facilitate use of different embodiments of tissue cutter device 10 in different locations of the body.

Some embodiments of device 10 may further include one or more electrodes coupled with platform 40 and/or flexible shaft portion 12c, for transmitting energy to tissues and thereby confirm placement of device 10 between target and non-target tissues. For example, electrodes may be placed on a lower surface of platform 40 and/or an upper surface of flexible shaft portion 12c, and the electrodes may be separately stimulated to help confirm the location of neural tissue relative to blades 26, 28. In such embodiments, nerve stimulation may be observed as visible and/or tactile muscle twitch and/or by electromyography (EMG) monitoring or other nerve activity monitoring. In various alternative embodiments, additional or alternative devices for helping position, use or assess the effect of tissue cutter device 10 may be included. Examples of other such devices may include one or more neural stimulation electrodes with EMG or SSEP monitoring, ultrasound imaging transducers external or internal to the patient, a computed tomography (CT) scanner, a magnetic resonance imaging (MRI) scanner, a reflectance spectrophotometry device, and a tissue impedance monitor disposed across a bipolar electrode tissue modification member or disposed elsewhere on tissue cutter device 10.

Wire bundle 24 may include as few as two wires and as many as one hundred or more wires. In various embodiments, each wire may be a solid wire, a braided wire, a core with an outer covering or the like, and may be made of any suitable material. For example, in various embodiments, wires of bundle 24 may be made from any of a number of metals, polymers, ceramics, or composites thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for the wires or for portions or coatings of the wires may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In some embodiments, all wires of bundle 24 may be made of the same material, whereas in alternative embodiments, wires may be made of different materials. Individual wires may also have any length, diameter, tensile strength or combination of other characteristics and features, according to various embodiments, some of which are discussed in greater detail below.

In various embodiments, wires of wire bundle 24 may be bound or otherwise coupled together at one or more coupling points or along the entire length of bundle 24. In one embodiment, for example, wires may be coupled together by a sleeve or coating overlaying bundle 24. In another embodiment, wires may only be coupled together at or near their proximal ends, at or near their connection point to tube 18, shaft 12, 14 or the like. In an alternative embodiment, wires may be individually coupled with an actuator, such as moveable handle 14, and not coupled to one another directly. In any case, wires will typically be able to move at least somewhat, relative to one another. This freedom of movement facilitates the change of cross-sectional shape that wire bundle 24 undergoes as it passes through differently shaped portions of shaft 12b, 12c. The change in cross-sectional shape of wire bundle 24 may convey different properties on device 10 at different portions, such as enhanced rigidity at one portion and enhanced flexibility at another. In some embodiments, wires may be individually coupled with a proximal actuator and may also be bound together at at least one point along their lengths. Optionally, the proximal actuator may allow one or more individual wires to be pulled, pushed and/or twisted, which acts to steer wire bundle 24 and thus steer a distal portion of device 10.

In some embodiments, wire bundle 24 may include one or more elongate, flexible members for performing various functions, such as enhancing tissue cutting, visualizing a target area or the like. For example, in various embodiments, bundle 24 may include an optical fiber, a flexible irrigation/suction tube, a flexible high pressure tubing, a flexible insulated tubing for carrying high temperature liquids, a flexible insulated tubing for carrying low temperature liquids, a flexible element for transmission of thermal energy, a flexible insulated wire for the transmission of electrical signals from a sensor, a flexible insulated wire for the transmission of electrical signals towards the distal end of the wires, an energy transmission wire, or some combination thereof. Examples of visualization devices that may be used include flexible fiber optic scopes, CCD (charge-coupled device) or CMOS (complementary metal-oxide semiconductor) chips at the distal end of flexible probes, LED illumination, fibers or transmission of an external light source for illumination or the like.

When blades 26, 28 face target tissue to be modified, such as buckled, thickened or otherwise impinging ligamentum flavum tissue, device 10 is configured such that platform 40 faces non-target tissue. Platform 40 may thus act as a tissue protective surface, and in various embodiments platform 40 may have one or more protective features, such as a widened diameter, protective or lubricious coating, extendable or expandable barrier member(s), drug-eluting coating or ports, or the like. In some instances, platform 40 may act as a “non-tissue-modifying” surface, in that it may not substantially modify the non-target tissue. In alternative embodiments, platform 40 may affect non-target tissue by protecting it in some active way, such as by administering one or more protective drugs, applying one or more forms of energy, providing a physical barrier, or the like.

Blades 26, 28 may be disposed on platform 40, with proximal blade being unattached to platform 40 and thus free to reciprocate with the back and forth movement of wire bundle 24, to which it is attached. Distal blade 28 is attached to platform 40 and thus remains stationary, relative to proximal blade 26 and wire bundle 24. In alternative embodiments, the distal end of wire bundle 24, itself, may be used to cut tissue, and device 10 may thus not include proximal blade 26. The distal end of wire bundle 24 may advance toward distal blade 28 to cut target tissue, or in alternative embodiments, wire bundle 24 may advance toward a non-sharp backstop to cut tissue or may simply advance against tissue to ablate it, without pinching the tissue between the wire bundle 24 distal end and any other structure. An example of the latter of these embodiments might be where ultrasound energy is used to reciprocate wire bundle 24, in which case the reciprocation of wire bundle 24 may be sufficient to cut or ablate tissue, without pinching or snipping between wire bundle and another structure.

In various embodiments, blades 26, 28, or other cutting structures such as the distal ends of wire bundle 24, a backstop or the like, may be disposed along any suitable length of shaft 12 and/or platform 40. In the embodiment shown in FIG. 5A, for example, blades 26, 28 are disposed along a length of platform 40. In an alternative embodiment, shaft 12 may comprise a hollow portion through which wire bundle 24 travels and a window through which wire bundle 24 is exposed. In any case, blades 26, 28 or other cutting members may be disposed or exposed along a desired length of device 10, to help limit an area in which the cutting members are active, thus helping to limit the exposure of non-target tissues to such cutting elements. In one embodiment, for example, such as an embodiment of the device to be used in a spinal treatment, blades 26, 28 may be disposed along a length of platform 40 measuring no longer than about 10 cm, and preferably no more than about 6 cm, and even more preferably no more than about 3 cm. In various embodiments, the length along which blades 26, 28 are disposed may be selected to approximate a length of a specific anatomical treatment area.

Blades 26, 28 may be made from any suitable metal, polymer, ceramic, or combination thereof. Suitable metals, for example, may include but are not limited to stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). In some embodiments, materials for blades 26, 28 or for portions or coatings of blades 26, 28 may be chosen for their electrically conductive or thermally resistive properties. Suitable polymers include but are not limited to nylon, polyester, Dacron®, polyethylene, acetal, Delrin® (DuPont, Wilmington, Del.), polycarbonate, nylon, polyetheretherketone (PEEK), and polyetherketoneketone (PEKK). In some embodiments, polymers may be glass-filled to add strength and stiffness. Ceramics may include but are not limited to aluminas, zirconias, and carbides. In various embodiments, blades 26, 28 may be manufactured using metal injection molding (MIM), CNC machining, injection molding, grinding and/or the like. Proximal and distal blades 26, 28 may be attached to wire bundle 24 and platform 40, respectively, via any suitable technique, such as by welding, adhesive or the like.

Tissue collection chamber 42 may be made of any suitable material, such as but not limited to any of the materials listed above for making blades 26, 28. In one embodiment, for example, chamber 42 may comprise a layer of polymeric material stretched between distal blade 28 and platform 40. In another embodiment, collection chamber 42 and distal blade 28 may comprise one continuous piece of material, such as stainless steel. Generally, distal blade 28 and chamber 42 form a hollow, continuous space into which at least a portion of cut tissue may pass after it is cut.

Guidewire connector 30 generally comprises a member build into or coupled with platform 40, at or near its distal tip, for coupling device 10 with a guidewire. For example, connector 30 may include a receptacle for accepting a ball tip of a guidewire and holding it to prevent unwanted guidewire release. In alternative embodiments, connector 30 may be replaced with a guidewire lumen or track for advancing device 10 over a guidewire.

With reference now to FIG. 5B, proximal handle 16 may be squeezed (hollow-tipped arrow) to advance moveable shaft portion 14, which thus pushes against wire bundle tube 18 to advance wire bundle 24 (solid-tipped arrow) and proximal blade 26. Handle 16 may then be released and squeezed again as many times as desired to cut a desired amount of tissue.

The advancement of proximal blade 26 is also depicted in FIGS. 5C and 5D. FIG. 5C is a top view of a portion of tissue cutter device 10, showing the multiple wires of wire bundle 24 and with blades 26, 28 in the open position. FIG. 5D shows the moveable shaft portion 14 advanced (hollow-tipped arrow) and wire bundle 24 and proximal blade 26 advanced to meet distal blade 28.

Referring to FIG. 5E, a cross-sectional view of a portion of device 10 demonstrates that wire bundle 24 assumes the cross-sectional shape of distal rigid shaft portion 12b where it is disposed in that portion and assumes the cross-sectional shape of flat flexible portion 12c where it is disposed in that portion. Thus, in some embodiments, wire bundle 24 may assume the cross-sectional shape of the shaft or other containing structure in which it resides.

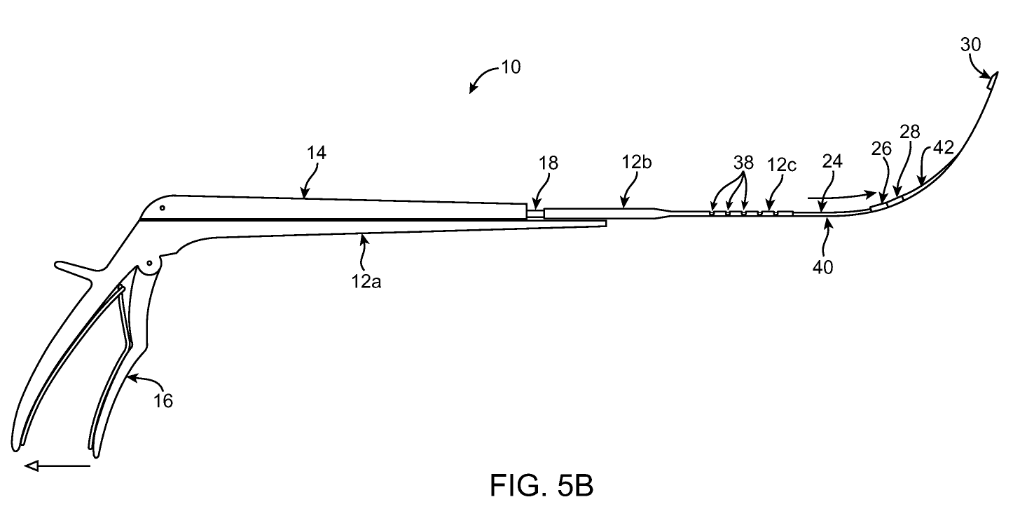

With reference now to FIG. 6, a portion of a tissue cutter device 50 is shown, in this embodiment including proximal shaft portion 52, a distal shaft portion 54 having multiple slits 56, and a wire bundle 58 disposed within shaft 52, 54. Each wire of bundle 58 includes a distal end 60 and a proximal end 62. This portion of device 50 shows in greater detail how in some embodiments wire bundle 58 may have a first cross-sectional configuration in one portion of shaft 52 and a second cross-sectional configuration in another portion of shaft 54. In fact, the cross-sectional shape of a portion of bundle 58 may change as that portion passes from proximal shaft portion 52 to distal shaft portion 54 or vice versa. Changing the cross-sectional shape of wire bundle 58 along the length of shaft 52, 54 may enhance flexibility of device 50 along one or more portions and/or may give one or more portions of device 50 an overall shape that facilitates its passage between closely apposed tissues, through a small channel, around a tight corner or the like. Wire bundle 58 will be disposed within shaft 52, 54 such that the individual wires of the bundle have at least some freedom to move relative to one another, thus enabling the cross-sectional shape of bundle 58 to change. In various alternative embodiments, wire bundle 58 may have any of a number of cross-sectional shapes, and may either change from one shape to another as it passes through shaft 52, 54 or, alternatively, may maintain the same shape throughout the length of an alternative shaft. As has been mentioned previously, further flexibility may be conferred on device 50 via slits 56.

In some embodiments, the changeability of the cross-sectional shape of wire bundle 58 may also be used to measure a contour or shape of an anatomical structure. For example, flexible bundle of wires 58 may be pressed against a contour to be measured, and bundle 58 may then be locked, to lock the cross-sectional shape of the contour into bundle 58. Device 50 may then be withdrawn from the patient, and the contour measured or otherwise assessed.

In some embodiments, rather than coupling the distal end of wire bundle 58 with a blade, distal ends 60 of the wires themselves may be used to cut tissue. Distal tips 60 may have any of a number of configurations, some of which are described in greater detail below. These ends 60 may be used to cut, scrape, pummel, chisel, shatter, ablate or otherwise modify tissue in various embodiments. In some embodiments, wire bundle 58 may be advanced and retracted using a manually powered handle to cut tissue with ends 60. Alternatively, as will be described further below, ends 60 may be reciprocated using ultrasound energy, using a rotational, powered driving mechanism, or the like.

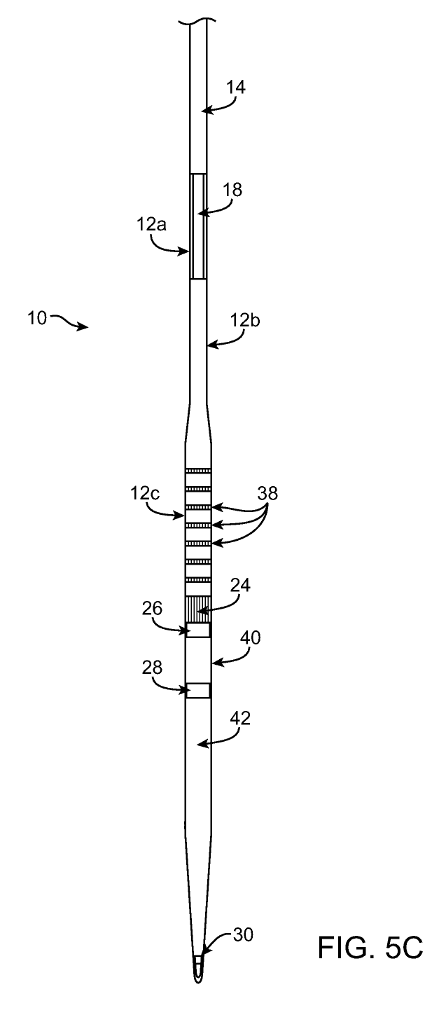

Referring to FIG. 7, a portion of an alternative embodiment of a tissue cutter device 70 may include a shaft 72 with a window 73 and a wire bundle 74 slidably disposed within shaft 72. The individual wires of bundle 74 may include distal tips 76, which may be sharpened in some embodiments. Wire bundle 74 may be reciprocated back and forth to cut tissue through window 73. In some embodiments, window 73 may include a sharpened edge 78, and tips 76 of wire bundle 74 may work with edge 78 to cut or snip off tissue. In an alternative embodiment, sharpened edge 78 may be left off, and distal tips 76 may advance tissue against a blunt or rounded edge of window 73.

As is evident from FIG. 7, in some embodiments, shaft 72 and wire bundle 74 may have a generally round cross-sectional shape. Such a configuration may be advantageous, for example, if shaft 72 is a flexible, elongate catheter. In some embodiments, the individual wires of wire bundle 74 may be free enough to move, relative to one another, that they can conform to a surface to be cut, such as a curved surface of a bone or the like. Such a shape conformation may facilitate even cutting of a tissue surface.

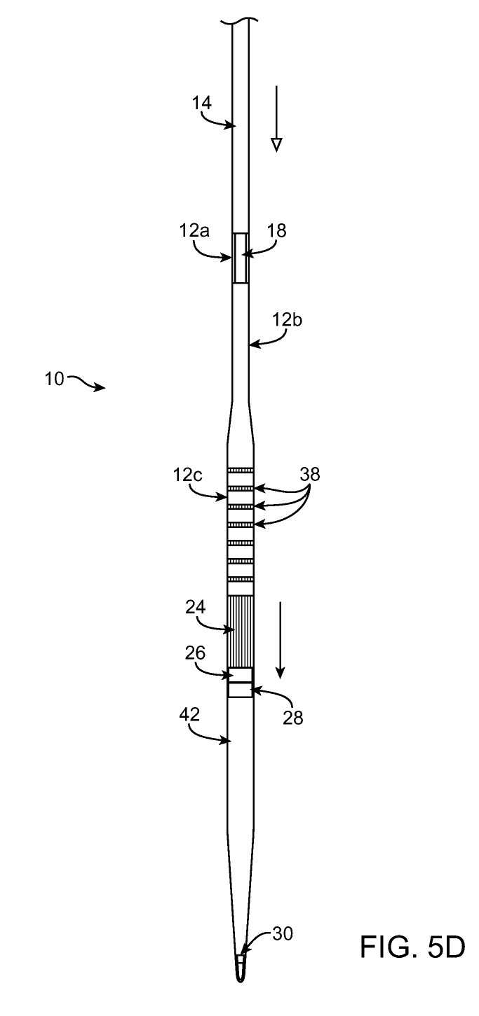

In an alternative embodiment, and with reference now to FIG. 8, a tissue cutter device 80 may include a shaft 82 with a window 83, a wire bundle 84 slidably disposed within shaft 82, a curved blade 86 coupled with the distal end of bundle 84, and a sharpened edge 88 of window 83. In an alternative embodiment, sharpened edge 88 may be left off, and blade 86 may advance tissue against a blunt or rounded edge of window 83.

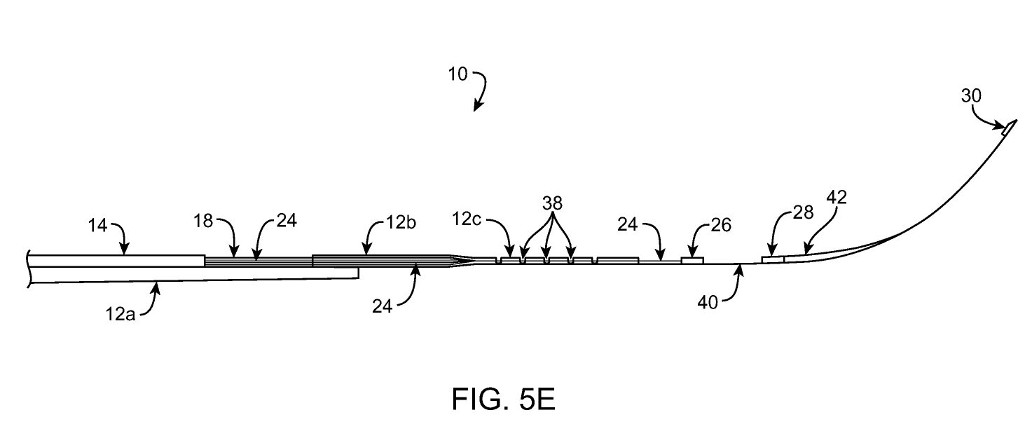

FIGS. 9A-9F show distal ends (or “tips”) of a variety of wires, which may be used to form wire bundles according to various embodiments of the tissue cutters described herein. These figures are provided for exemplary purposes only, and other embodiments of wires may have alternative shapes. In the embodiments shown, a wire may have a beveled tip 92 (FIG. 9A), double-beveled tip 94 (FIG. 9B), flat/squared-off tip 96 (FIG. 9C), rounded tip 98 (FIG. 9D), inverted double-bevel tip 100 (FIG. 9E), or bent/scraper tip 102 (FIG. 9F). Additionally, various wires may have any desired diameter, length, tensile strength or cross-sectional shape. For example, a typical wire may have a round cross-sectional shape, but alternative wires may have oval, square, rectangular, triangular, hexagonal or other cross-sectional shapes.

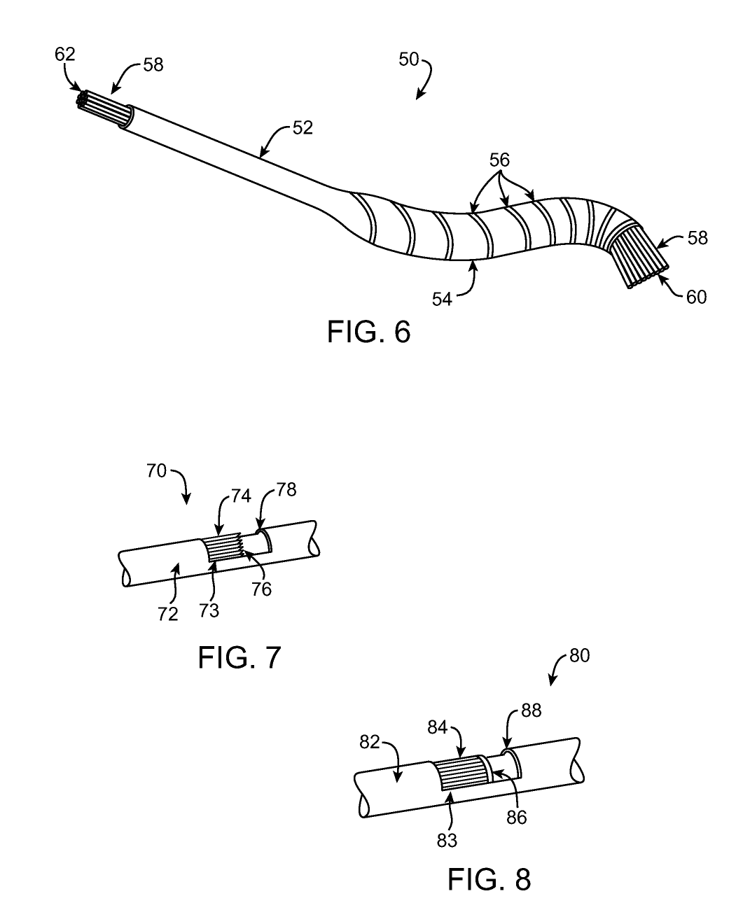

Referring now to FIGS. 10A-10G, just as wires may have different tip shapes in different embodiments, shafts and wire bundles may have different cross-sectional shapes in different embodiments. Typically, the cross-sectional shape of a shaft will determine the cross-sectional shape of a wire bundle that passes through it, since the wires of the bundle will be at least somewhat free, relative to one another. As has been described above, in various embodiments, a shaft may have one cross-sectional shape along its entire length or, alternatively, it may have two or more different cross-sectional shapes, such as a round shape proximally and a flatter shape distally. The embodiments shown, which are merely examples, include a round shaft 104 with a round wire bundle 105 (FIG. 1A), a square shaft 106 with a square wire bundle 107 (FIG. 10B), a rectangular shaft 108 with a rectangular wire bundle 109 (FIG. 1C), an oval shaft 110 with an oval wire bundle 111 (FIG. 10D), a flat shaft 112 with a flat wire bundle 113 (FIG. 10E), an asymmetric shaft 114 with an asymmetric wire bundle 115 (FIG. 10F), and a V-shaped shaft 116 with a V-shaped wire bundle 117 (FIG. 10G). Any of these shapes or other shapes may be used alone or in combination in any given embodiment of a multi-wire tissue cutter device.

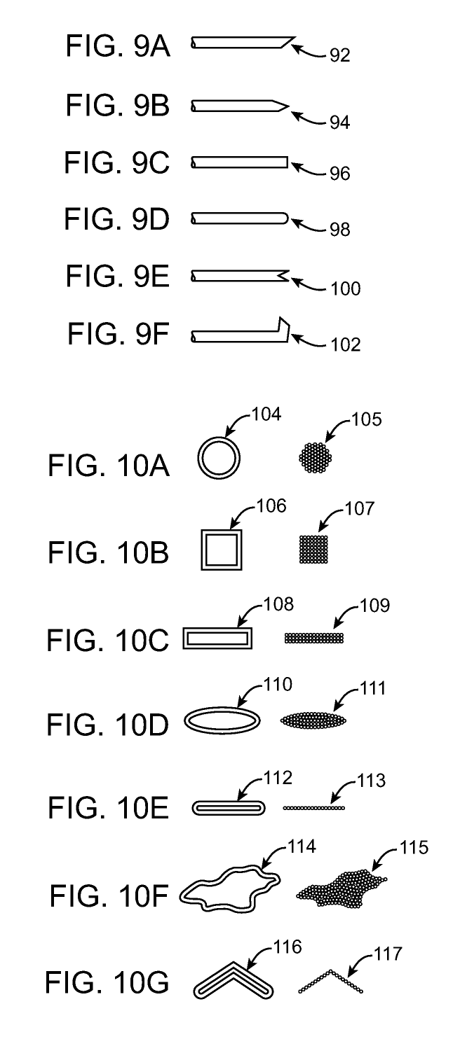

With reference now to FIGS. 11A and 11B, in one embodiment, a tissue cutter device 120 (only a portion of which is shown) may include a shaft 122 having multiple slits 124 for flexibility and a window 126, and multiple cutting members, which may be advanced into window 126 to cut tissue. In some embodiments, for example, it may be advantageous to have one or more cutting members for cutting soft tissue, such as ligament, and one or more cutting members for cutting hard tissue, such as bone. For example, in one embodiment, referring to FIG. 11A, a distal blade 128 may be advanced (hollow-tipped arrow) and used to cut soft tissue, such as ligament. Blade 128 may then optionally be retracted back into shaft 122, and (referring to FIG. 11B) a wire bundle cutting member 130 may be advanced (solid-tipped arrow) to cut bone. In one embodiment, for example, distal blade 128 may be used to cut tissue by manually moving shaft back and forth to caused blade 128 to slice tissue, while wires 130 may be reciprocated rapidly, such as by ultrasound power, to ablate or pulverize bone.

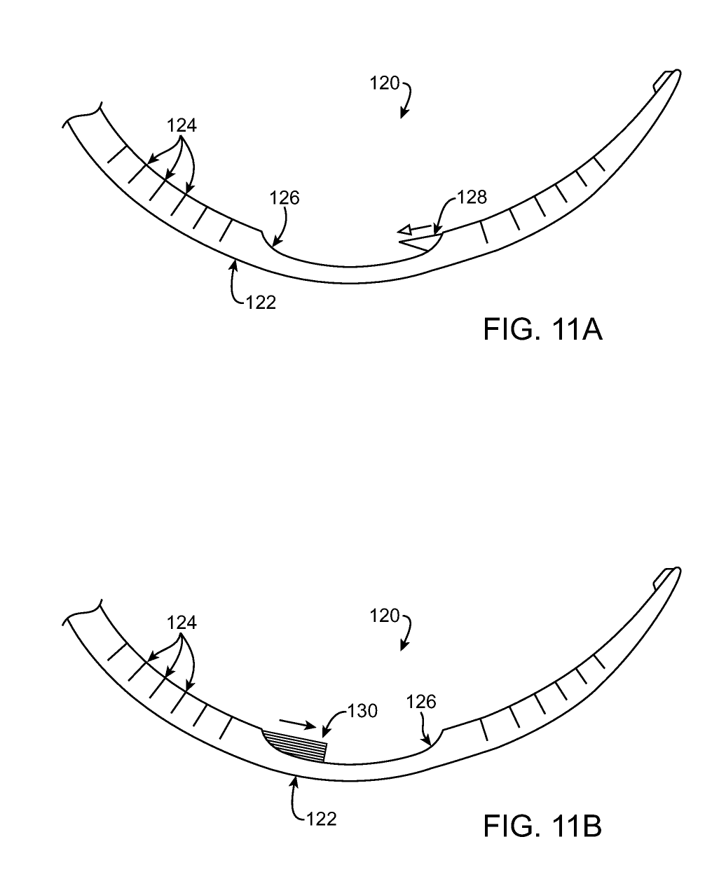

Referring to FIGS. 12A and 12B, in another alternative embodiment, a tissue cutter device 140 (only a portion of which is shown) may include a stationary shaft portion 142 having a window 144, a moveable shaft portion 143, a wire bundle 146, and a ramp 147 and plateau 148 coupled with an inner surface of moveable portion 143. When moveable portion 143 is placed in a first position, ramp 147 deflects a distal end of wire bundle 146 out of window 144 to facilitate tissue removal, such as of soft tissue, and to control the depth of tissue cut. Moveable portion 143 may be repositioned (FIG. 12B, hollow-tipped arrow) to bring ramp within stationary shaft 142, such that wire bundle 146 is not deflected out of window 144 but instead travels forward in a relatively straight direction over plateau 148. Reciprocating wire bundle 146 back and forth in a relatively straight path may be advantageous for cutting hard tissue, such as bone.

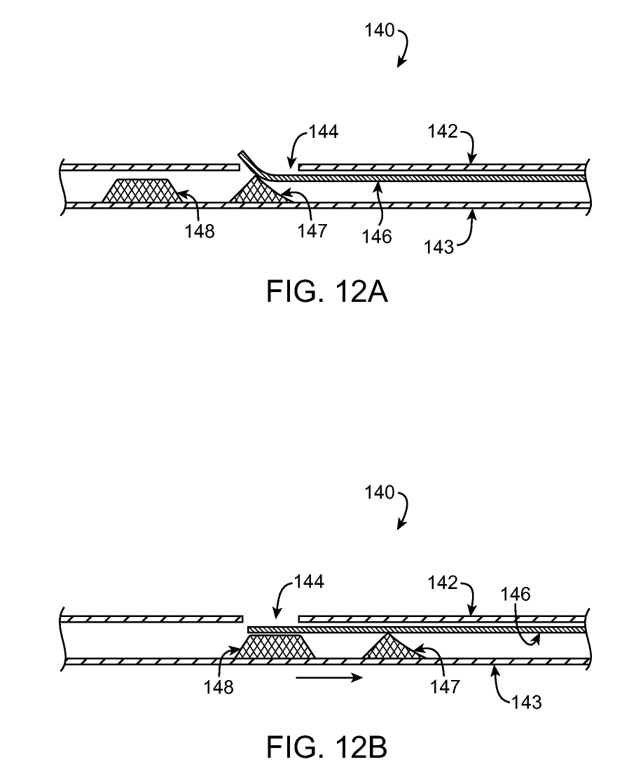

In an alternative embodiment, as shown in FIG. 13, a tissue cutter device 150 may be configured similarly to the embodiment shown in FIGS. 5A-5E but may further include a radiofrequency (RF) wire loop cutter 168. As in the earlier-described embodiment, cutter device 150 may include a movable shaft portion 154, a proximal stationary shaft portion 152a, a distal stationary shaft portion 152b, and a flexible shaft portion 152c having multiple slits 160 for enhanced flexibility. Device 150 may also include a wire bundle tube 158 into which a proximal end of a wire bundle 161 is secured, a proximal blade 162 coupled with the distal end of wire bundle 161, a distal blade 164, and a guidewire connector 166. In addition, in one embodiment, device 150 may further include RF wire loop 168, which may optionally be retractable into shaft 152c. RF energy may be applied to loop cutter 168, for example, for cutting soft tissue such as ligament. Blades 162, 164 may be used to cut additional soft tissue and/or to cut bone.

Wire loop 168 may comprise any suitable RF electrode, such as those commonly used and known in the electrosurgical arts, and may be powered by an internal or external RF generator, such as the RF generators provided by Gyrus Medical, Inc. (Maple Grove, Minn.). Any of a number of different ranges of radio frequency may be used, according to various embodiments. For example, some embodiments may use RF energy in a range of between about 70 hertz and about 5 megahertz. In some embodiments, the power range for RF energy may be between about 0.5 Watts and about 200 Watts. Additionally, in various embodiments, RF current may be delivered directly into conductive tissue or may be delivered to a conductive medium, such as saline or Lactated Ringers solution, which may in some embodiments be heated or vaporized or converted to plasma that in turn modifies target tissue. In various embodiments, wire loop 168 may be caused to extend out of a window of a shaft, expand, retract, translate and/or the like. One or more actuators (not shown) for manipulating and/or powering wire loop 168 will typically be part of device 150 and may either be coupled with, integrated with or separate from an actuator for reciprocating wire bundle 161.

The embodiment shown in FIG. 13 is only one example of how, in some embodiments, multi-wire tissue cutter device 150 may employ two or more different cutting modalities in the same device. For example, one tissue cutter device may include, in addition to a multi-wire bundle, any one or more of such tissue manipulation devices as a rongeur, a curette, a scalpel, a scissors, a forceps, a probe, a rasp, a file, an abrasive element, a plane, a rotary powered mechanical shaver, a reciprocating powered mechanical shaver, a powered mechanical burr, a laser, an ultrasound crystal a cryogenic probe, a pressurized water jet, a drug dispensing element, a needle, a needle electrode, or some combination thereof. In some embodiments, for example, it may be advantageous to have one or more tissue modifying members that stabilize target tissue, such as by grasping the tissue or using tissue restraints such as barbs, hooks, compressive members or the like. In one embodiment, soft tissue may be stabilized by applying a contained, low-temperature substance (for example, in the cryo-range of temperatures) that hardens the tissue, thus facilitating resection of the tissue by a blade, rasp or other device. In another embodiment, one or more stiffening substances or members may be applied to tissue, such as bioabsorbable rods.

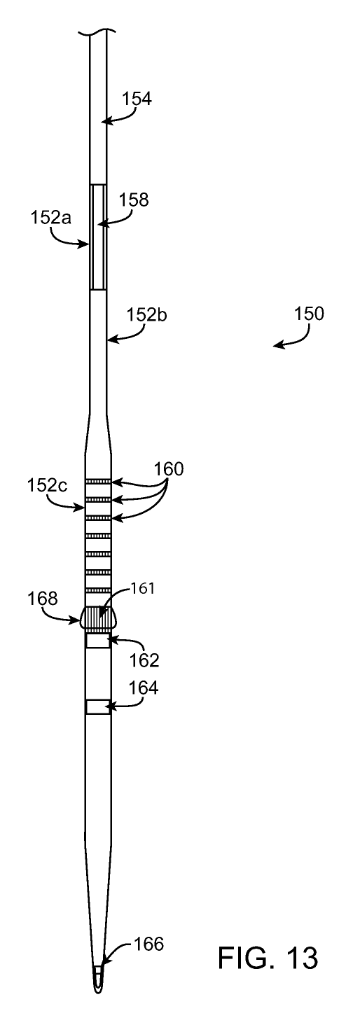

With reference now to FIG. 14, in another embodiment, a multi-wire tissue cutter device 190 may include a proximal handle 192 with an actuator 193, a rigid shaft portion 194 extending from handle 192, an elongate flexible shaft portion 198 extending from rigid shaft 194 and having a window 199, and a wire bundle 196 extending through flexible shaft 198 and into window 199. In various embodiments, rigid portion 194 and flexible portion 198 may have any desired lengths. When actuator 193 is squeezed and released (hollow-tipped, double-headed arrow), a driving mechanism in rigid shaft portion 194 reciprocates (solid-tipped, double-headed arrow), thus causing wire bundle 196 to reciprocate (open, double-tipped arrow) to cut or otherwise ablate tissue.

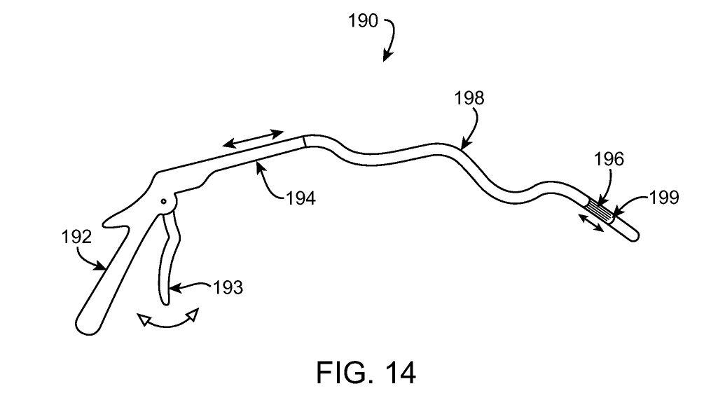

FIG. 15 shows another embodiment of a multi-wire tissue cutter device 170, including a motor 172, a drive shaft 174, an at least partly flexible shaft 178 having a window 179, and a wire bundle 176 slidably disposed within shaft 178 and extending into window 179 to cut tissue. Generally, motor 172 rotates about a central axis (solid-tipped arrow) to cause drive shaft 174 to reciprocate (hollow-tipped, double-headed arrow), thus moving wires back and forth through shaft 178. At least a proximal portion of shaft 178 remains stationary (diagonal lines), relative to drive shaft 174, so that wire bundle 176 moves through shaft.

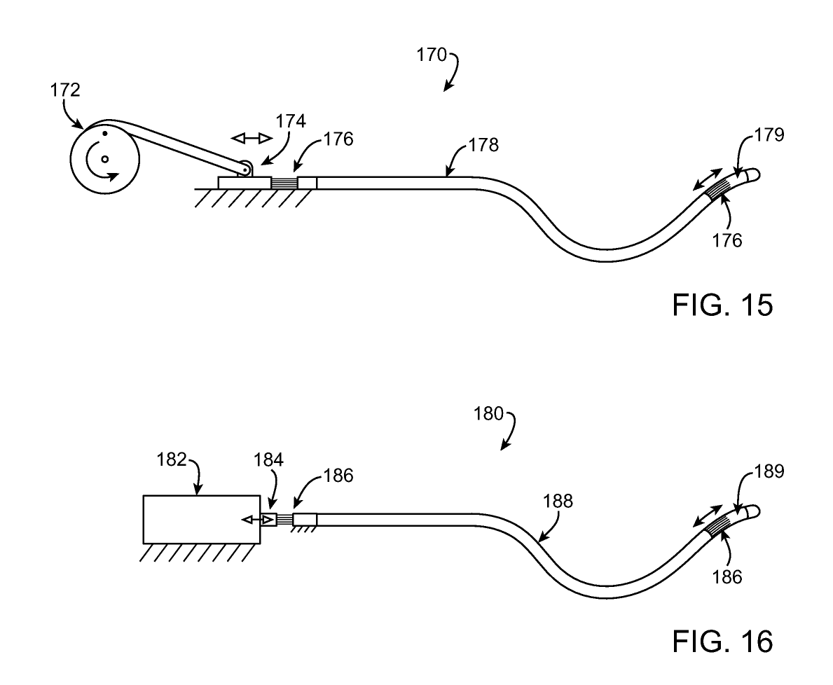

In another embodiment, and with reference now to FIG. 16, a tissue cutter device 180 may include an ultrasound source 182, a drive shaft 184 coupled with source 182, a wire bundle 186 coupled with drive shaft 184, and an at least partly flexible shaft 188 with a window 189. In this embodiment, ultrasound source 182 and a proximal portion of shaft 188 (such as a proximal handle or the like) remain stationary, and drive shaft 184 reciprocates (hollow-tipped, double-headed arrow) to reciprocate wire bundle 186 through shaft 188. The distal end of wire bundle 186, reciprocated at ultrasonic frequencies, may be used to cut or ablate soft tissue and/or bone. In various alternative embodiments, other alternative mechanisms for driving a bundle of wires, such as gears, ribbons or belts, magnets, electrically powered, shape memory alloy, electro magnetic solenoids and/or the like, coupled to suitable actuators, may be used.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. These and many other modifications may be made to many of the described embodiments. Therefore, the foregoing description is provided primarily for exemplary purposes and should not be interpreted to limit the scope of the invention as it is set forth in the claims.

We claim:

1. A device for cutting tissue in a human body, the device comprising:
an elongate, hollow shaft having a proximal portion and a distal portion;
a bundle of flexible wires slidably disposed within at least a portion of the shaft and having a proximal end and a distal end, wherein the distal end of the bundle is configured to facilitate cutting of tissue, and wherein the wires of the bundle are at least partially free to move, relative to one another, to allow a cross-sectional shape of the bundle to differ along a length from the proximal to the distal end; and
an actuator coupled with the proximal portion of the shaft and the proximal end of the bundle of wires, wherein the actuator is configured to move the wires back and forth through the hollow shaft to cause the distal ends of the wires to cut tissue.

2. A device as in claim 1, wherein the shaft has at least one cross-sectional shape selected from the group consisting of round, square, triangular, oval, elliptical, flat, rectangular, asymmetrical, triangular, v-shaped and w-shaped.

3. A device as in claim 1, wherein the proximal portion of the shaft has a first cross-sectional shape, and the distal portion of the shaft has a second cross-sectional shape, and wherein the bundle of wires assumes approximately the first cross-sectional shape in the proximal portion and approximately the second cross-sectional shape in the distal portion.

4. A device as in claim 1, wherein the shaft proximal portion is rigid and the shaft distal portion is at least partially flexible.

5. A device as in claim 4, wherein the flexible distal portion is steerable, the device further comprising at least one shaft steering actuator.

6. A device as in claim 1, wherein the shaft further comprises at least one window through which tissue may protrude such that the wires may cut the protruding tissue.

7. A device as in claim 6, wherein the shaft includes at least one hollow tissue collection chamber beyond the window.

8. A device as in claim 6, wherein window includes a blade edge, and wherein the wire bundle is configured to push tissue against the blade edge.

9. A device as in claim 6, further comprising a slidable ramp member disposed within the shaft for sliding into contact with the wire bundle to urge at least some of the wires out the window to cut tissue and control a depth of the cut.

10. A device as in claim 1, wherein the distal portion of the shaft includes a distal opening, and wherein the wire bundle extends out of the distal opening to cut tissue.

11. A device as in claim 10, further comprising a flexible platform extending beyond the distal opening in the shaft, wherein the platform extends under the wires to protect non-target tissue.

12. A device as in claim 1, wherein the wires comprise a material selected from the group consisting of nitinol, spring stainless steel and other metallic spring materials.

13. A device as in claim 1, wherein the wires are coupled together along at least a portion of their lengths.

14. A device as in claim 1, wherein the wires are uncoupled to one another.

15. A device as in claim 1, wherein the proximal end of each wire includes a coupling member or shape to attach to the actuator, and wherein each wire is individually attached to the actuator.

16. A device as in claim 1, further including a blade coupled with the distal end of the bundle of wires to cut the tissue.

17. A device as in claim 16, wherein the blade is coupled with the distal end of individual wires in the bundle of wires via individual separate hinges, at separate locations on the blade, such that the blade may move from a first configuration substantially parallel to the path of the wires to a second configuration at an angle to the path of the wires, by separately moving one or more wires coupled with the blade.

18. A device as in claim 16, wherein a window on the shaft includes a blade edge, and wherein the blade coupled with the bundle of wires moves toward the blade edge on the window to cut tissue.

19. A device as in claim 1, wherein the actuator is selected from the group consisting of a squeezable handle, a handle with a trigger, an ultrasound transducer, and a rotary driven reciprocating device.

20. A device as in claim 1, wherein the actuator is configured to at least one of pull, push and twist at least one individual wire of the bundle, and wherein the wires are at least partially coupled together, such that the actuator can steer the bundle by manipulating the individual wire(s).

21. A device as in claim 1, wherein the bundle of wires further comprises at least one of an optical fiber, a flexible irrigation/suction tube, a flexible high pressure tubing, a flexible insulated tubing for carrying high temperature liquids, a flexible insulated tubing for carrying low temperature liquids, a flexible element for transmission of thermal energy, a flexible insulated wire for the transmission of electrical signals from a sensor, a flexible insulated wire for the transmission of electrical signals towards the distal end of the wires and an energy transmission wire.

22. A method for cutting tissue in a human body, the method comprising:
advancing an elongate, hollow shaft of a tissue cutting device at least partway into the body such that a tissue cutting portion of the device faces target tissue and a non-cutting portion of the device faces non-target tissue; and
advancing a bundle of flexible, elongate wires longitudinally through the hollow shaft to cut at least a portion of the target tissue using distal ends of the wires.

23. A method as in claim 22, wherein advancing the shaft comprises pulling the shaft into place between target and non-target tissue by pulling a guidewire coupled with a distal end of the shaft.

24. A method as in claim 22, wherein advancing the shaft comprises advancing over a guidewire.

25. A method as in claim 22, wherein advancing the shaft comprises positioning a window of the shaft against the target tissue.

26. A method as in claim 22, wherein advancing the shaft comprises steering at least a distal, flexible portion of the shaft.

27. A method as in claim 22, wherein advancing the wires comprises pulling a squeeze handle of a proximal actuator coupled with proximal ends of the wires.

28. A method as in claim 22, wherein advancing the wires comprises activating an ultrasound transducer coupled with proximal ends of the wires.

29. A method as in claim 22, wherein advancing the wires comprises activating a rotary reciprocating actuator coupled with proximal ends of the wires.

30. A method as in claim 22, wherein advancing the wires causes the bundle to change its cross-sectional shape as it passes through differently shaped portions of the shaft.

31. A method as in claim 22, wherein advancing the wires causes at least some of the wires to pass by a window on the shaft to cut tissue protruding through the window.

32. A method as in claim 31, wherein advancing the wires causes some of the wires to extend out of the window.

33. A method as in claim 31, wherein advancing the wires urges tissue against a sharpened edge of the window to cut tissue.

34. A method as in claim 22, wherein advancing the wires causes distal ends of the wires to extend out of a distal opening of the shaft.

35. A method as in claim 22, wherein advancing the wires causes the wires to separate at their distal ends.

36. A method as in claim 22, wherein the distal ends of the wires are coupled with a blade, and wherein advancing the wires causes the blade to cut tissue.

37. A method as in claim 22, wherein the wires automatically retract after being advanced.

38. A method as in claim 22, further comprising reciprocating the wires back and forth multiple times.

39. A method as in claim 22, wherein advancing the wires causes at least some cut tissue to pack into a hollow chamber of the shaft.

40. A method as in claim 22, further comprising visualizing the target tissue with an optical fiber disposed in the bundle of wires.

41. A method as in claim 22, further comprising introducing and/or suctioning fluid using a flexible tube disposed in the bundle of wires.

42. A method as in claim 22, further comprising delivering energy at the distal end of the bundle of wires, using a flexible energy delivery device disposed in the bundle.

43. A method as in claim 22, further comprising delivering fluid under high pressure at the distal end of the bundle of wires, using a fluid delivery tube disposed in the bundle.

44. A method as in claim 22, further comprising transmitting electrical signals from a sensor in the distal end of the bundle of wires, using a flexible insulated wire disposed in the bundle.

45. A system for cutting tissue in a human body, the system comprising:
a tissue cutting device, comprising:
an elongate, hollow shaft having a proximal portion with a first cross-sectional shape and a distal portion with a second cross-sectional shape;
a bundle of flexible wires slidably disposed within at least a portion of the shaft, each of the wires comprising a proximal end and a distal end, the distal end configured to facilitate cutting of tissue, wherein the wires are sufficiently free to move, relative to one another, to allow a cross-sectional shape of the bundle of wires to change from the first cross-sectional shape of the shaft proximal portion to the second cross-sectional shape of the shaft distal portion; and
an actuator coupled with the shaft and the bundle of wires at or near their proximal ends, wherein the actuator is configured to move the wires back and forth through the hollow shaft to cause the distal ends of the wires to cut tissue; and
a power source removably coupled with the actuator to provide power to move the wires back and forth.

46. A system as in claim 45, wherein the actuator comprises an ultrasound transducer, and wherein the power source comprises an ultrasound generator.

47. A system as in claim 45, wherein the actuator comprises a rotary driven reciprocating device, and wherein the power source comprises an electrical power source.

48. A system as in claim 45, wherein the actuator comprises a handle.

49. A system as in claim 48, wherein the power source is removably coupled with the handle.
